(19) United States
(12) Patent Application Publication
(10) Pub. No.: US 2011/0288381 A1
(43) Pub. Date: Nov. 24, 2011

(54) System And Apparatus For Correlating Heart Rate To Exercise Parameters

(76) Inventors: Jesse Bartholomew, Madison, WI (US); Allen C. Lim, Boulder, CO (US); Jeffrey T. Ivereson, Madison, WI (US)

(21) Appl. No.: 13/113,767

(22) Filed: May 23, 2011

Related U.S. Application Data
(60) Provisional application No. 61/347,716, filed on May 24, 2010.
(60) Provisional application No. 61/363,500, filed on Jul. 12, 2010.

(51) Int. Cl. A61B 5/0205; A61B 5/024; A61B 5/00

(52) U.S. Cl. 600/301; 600/300; 600/508

(57) ABSTRACT

A computer-implemented method is used for estimating an exercise parameter for a user. The method includes the steps of receiving a measured physiologic value such as heart rate, determining an estimated exercise parameter value based on the physiologic value based on a stored correlation between a measured physiologic value and a measured exercise parameter value, and providing the estimated exercise parameter value.

### CROSS REFERENCE TO RELATED APPLICATIONS

The present application claims priority to U.S. Provisional Patent Application No. 61/347,716, filed on May 24, 2010, and U.S. Provisional Patent Application No. 61/363,500, filed on Jul. 12, 2010, the entire disclosures of which are hereby incorporated by reference.

### BACKGROUND

This application relates to a method and apparatus for estimating one or more exercise parameters based on a measured physiologic value. More specifically, the present application is directed to an apparatus and method adapted to provide a user with an estimated value of one or more different exercise parameters based on a sensed heart rate.

Measuring exercise parameters during a training activity such as bicycling, for example, is a generally accepted means for assessing a user's fitness level and/or efficiency based on expended energy. Exercise parameters or exercise parameter values as used in this application can refer to any measurable physiological or exercise-related characteristics. For instance, oxygen consumption, carbon dioxide exhalation, and power output are all examples of measurable exercise parameters that are measured using a device adapted for detecting and measuring the particular value either instantaneously or over the course of a particular workout or exercise session.

Non-physiological measurements or characteristics may also be used in analyzing an exercise session or to provide information to a user during an exercise session. For instance, speed, slope of a particular trail or track, the resistance setting for a piece of exercise equipment, temperature, wind speed, etc. are examples of such non-physiological exercise parameters.

A user can use exercise parameter measurements to assess their performance during a given race or exercise or training session, to analyze performance after-the-fact, to plan future events, workouts or sessions, etc. The user can also compare the measurements of a given session to any number of previous or subsequent sessions such that he or she has an objective means by which to gauge his or her relative performance. Monitoring exercise parameters is particularly useful in training activities such as cycling and running sports where bicyclists and runners are training in variable conditions, such as courses of varying length and terrain, different weather conditions, different drafting partners, etc. Given the variations, it can be difficult to gauge performance from one session to the next unless the user repeatedly uses the same course under substantially the same conditions.

In order to track performance in variable conditions, many users employ specialty monitoring devices for measuring their performance during a given session. For example, power meters are one known device commonly employed by bicyclists to measure their power output during a ride. Power meters use one or more sensors to generate power output data and typically communicate with a handlebar mounted computer that displays the power output data along with other relevant data, such that a user may instantaneously see his or her instantaneous power output, maximum power output, average power output, etc.

Power meters come in a number of forms, and commonly utilize strain gauges advantageously deployed at particular locations on the bicycle for measuring the torque applied by the rider. When the torque is combined with a measured angular velocity, the power output of the rider may be determined. Commonly, strain gauges may be mounted in a bottom bracket, wheel hubs, pedals, shoes, or crankset of a bicycle. Instead of using strain gauges, other power meters generate power output data using handle-bar mounted units that measure opposing forces, such as gravity, wind resistance, inertia and rolling resistance, and combine these measurements with velocity to determine a rider's power output.

However, devices for accurately measuring exercise parameters can be expensive and/or not flexible enough to use in a variety of situations. For example, the power meters described above typically require affixed hardware that is not easily portable. This lack of portability is inconvenient since many cyclists use a number of different bicycles for training and/or racing, and thus require a separate power meter for each bicycle or a power meter in a component that can be moved from one bicycle to another. Power meters, however, are quite expensive thus rendering it very costly or cost prohibitive to own more than one power meter. Although some power meters are configured to be portable, transferring the power meter can involve significant effort and inconvenience, and sometimes a loss in performance. In another example, in a sport such as swimming, constraints on the type of equipment that can be used also make it difficult to ascertain a user's power output.

In addition to power output, a number of additional exercise parameters are useful in assessing an individual's performance during a given exercise or training session. For instance, measures of oxygen consumption and carbon dioxide exhalation are indicative of an individual's pulmonary performance during a given training activity. While oxygen consumption and carbon dioxide exhalation are quite useful parameters for assessing an individual's performance, it is not easy to measure these parameters. Typically, oxygen consumption and carbon dioxide exhalation are measured in a laboratory environment in which the individual is connected to a device specifically configured for measuring oxygen input and carbon dioxide output while the individual engages in a particular training activity, such as running on a treadmill or cycling on a stationary exercise cycle. Of course, such measurements are not practically obtainable during normal day-to-day exercise, and as such, individuals are incapable of easily and inexpensively assessing their pulmonary performance during exercise.

Other useful parameters include, but are not limited to, energy expended, which is typically represented as KiloJoules, and fuel (food) consumed, which is typically represented as Calories.

Accordingly, it is desired to provide an exercise parameter measuring system that is substantially low cost and portable. Further, it is generally recognized that other exercise activities would benefit from power measurement in the same manner as bicycling, and thus, it is desired to provide a power measuring system that may be used, not only in bicycling, but in any number of other exercise activities.

### SUMMARY

In one exemplary embodiment, a computer-implemented method is used for estimating an exercise parameter for a user. The method includes the steps of receiving a measured heart rate value, determining an estimated exercise parameter value based on the heart rate value using a stored correlation between a measured heart rate value and a measured exercise parameter value, and providing the estimated exercise parameter value.

In another exemplary embodiment, the present invention contemplates a computer system for providing an estimated exercise parameter value. The system includes a heart rate sensor input device, a processor configured to receive heart rate information from the heart rate input device and that executes a program that includes a correlation between the received heart rate information and estimated values for one or more exercise parameters, and an estimated exercise parameter output device.

The system may be configured such that the estimated exercise parameter output device is configured to transmit the received heart rate information. The system may further include memory for receiving and storing a measured correlation between the received heart rate information and exercise parameter values for a specific user.

According to yet another exemplary embodiment, a computer implemented method for generating a correlation between a measured heart rate and measured power output for a user during exercise is provided. The method includes receiving a measured heart rate value, receiving a measured power output value and determining a user-specific correlation between heart rate and power output. The correlation may be based on a regression analysis of the measured heart rate value and the measured power output value. The method further includes storing the user-specific correlation in computer memory. Determining a user-specific correlation between heart rate and power output may include determining a linear correlation between heart rate and power output, determining a first order correlation between the heart rate and power output, determining a linear correlation, a first order correlation, and a second order correlation between the heart rate and power output, and/or applying a differential correlation between the measured heart rate and power output.

In yet another exemplary embodiment, a computer-implemented method for estimating power output by a user includes the steps of receiving a measured heart rate value, determining an estimated power output based on the heart rate value using a stored correlation between a measured heart rate value and a power output value, and providing the power output value. The method may include receiving a user specific data entry wherein determining an estimated power output value based on the heart rate value includes customizing the estimated power output value based on the user specific data, wherein the user specific data is at least one of a gender, age, height, weight, fitness level, and fatigue level. The method may also be configured such that the stored correlation between a measured heart rate value and a power output value is generated based on a regression analysis of received measured heart rate values and power output exercise parameter values.

### BRIEF DESCRIPTION OF THE DRAWINGS

Preferred exemplary embodiments of the invention are illustrated in the accompanying drawings in which like reference numerals represent like parts throughout.

In the drawings:

FIG. 1 is a block diagram view of a system for generating and storing a correlation between a measured physiologic value and at least one measured exercise parameter;

FIG. 2 is a flowchart illustrating a method for generating and storing a correlation between a measured physiologic value and at least one measured exercise parameter;

FIG. 3 is a graph illustrating received heart rate data from a monitor and power output data from a monitor, generated using the system of FIG. 1;

FIG. 4 is a graph depicting the measured physiologic value against the measured exercise parameter value illustrating the data used by a regression engine to generate correlation information, generated using the system of FIG. 1;

FIG. 5 is a graph illustrating the differential calculation plots for power output over time, generated using the system of FIG. 1;

FIG. 6 is a schematic view of a bicycle including a computer configured to display an estimated exercise parameter generated based on a measured physiologic value; and

FIG. 7 is an exemplary computer configured to generate one or more estimated exercise parameters based on a detected physiologic value.

### DETAILED DESCRIPTION

The present invention contemplates a method and apparatus for providing a user with an estimate of one or more exercise parameters during a physical activity. The method of the invention includes developing a correlation between a measured heart rate reading and one or more measured exercise parameters. Once the correlation between the actual heart rate and actual exercise parameter is established, a regression analysis may be performed to determine a correlation between a measured heart rate and the measured exercise parameter. In other words, by correlating actual measured heart rate with an actual measured exercise parameter, the method and apparatus of the invention may use the derived correlation to thereafter estimate the one or more exercise parameters based on actual heart rate information. Thus, after the correlation is created, the user need not employ the use of a means for actually measuring the particular exercise parameter, but rather, he or she may simply use his or her heart rate information to provide the user with an estimate of his or her exercise parameter, either real-time or after the physical activity is concluded.

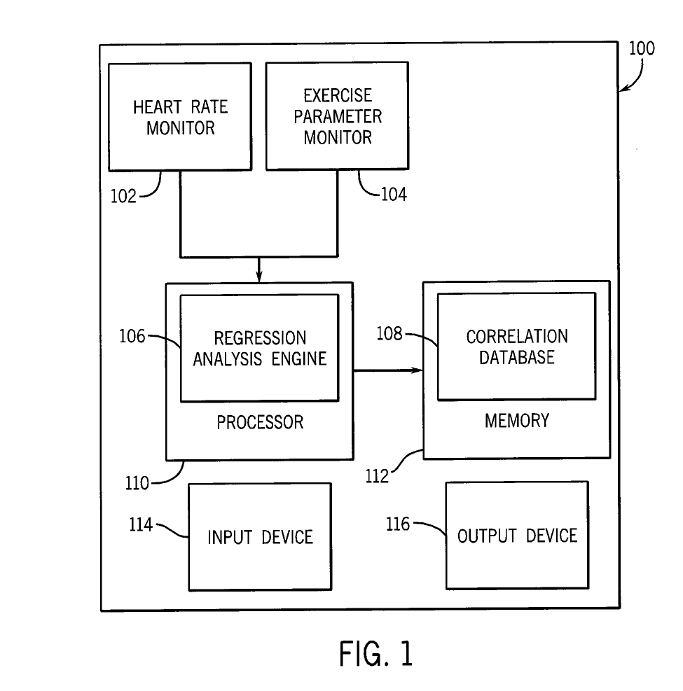

Referring now to FIG. 1, a calibration system 100 configured to receive measured physiologic data in combination with one or more measured exercise parameters to develop a correlation is shown, according to an exemplary embodiment. Calibration system 100 is configured to include a heart rate monitor 102, at least one exercise parameter monitor 104, a regression analysis engine 106, and a correlation database 108. Calibration system 100 may be implemented using a standard computer system including a processor 110, memory 112, one or more input devices 114 and one or more output devices 116. Although monitors 102 and 104 are shown as components to system 100, these systems may be implemented as external devices configured to provide generated monitoring data to system 100 through a wired or wireless connection.

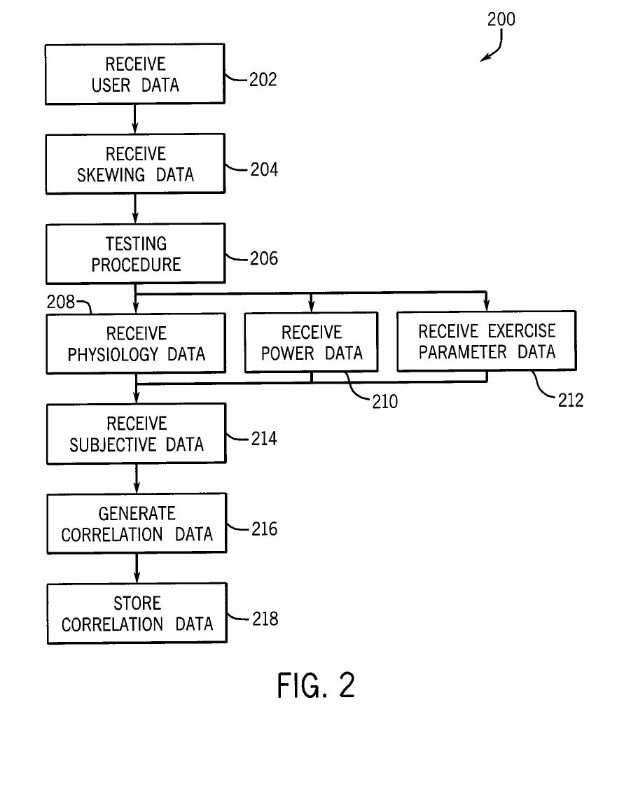

Referring now also to FIG. 2, a flowchart 200 illustrating a computer-implemented method for generating correlation data between a measured physiologic data value and one or more measured exercise parameters is shown, according to an exemplary embodiment. The method of flowchart 200 may be implemented by system 100. Although flowchart 200 illustrates a number of steps performed in a specific order, it should be understood that the method may be performed using more, fewer, and/or a different ordering of steps to implement the functionality described herein. Further, although specific steps are described herein as being performed by specific hardware, it should be understood that the steps may be performed by any type of hardware to implement the functions described herein.

In a step 202, system 100 is configured to receive user data. User data may be obtained based on measured values, such as data received from monitors 102 and 104 and/or may be manually entered using one or more input devices 114, such as a keyboard or mouse. Exemplary user data includes, but is not limited to, height, weight, gender, age, relative fitness, maximum heart rate, resting heart rate, etc.

In a step 204, system 100 is configured to identify and receive any correlation skewing factors. Exemplary correlation skewing factors can include, but are not limited to, determining whether a testing procedure is to be performed inside or outside, determining current temperature, determining training course profile, determining recent caloric activity by the user, determining recent activity by the user, determining type of monitoring devices, etc. The correlation skewing factors may be determined based on a questionnaire, uploaded data, data from one or more sensors, etc.

In a step 206, a testing procedure is performed to generate a measured physiologic value over time and one or more measured exercise parameters over time using the monitors 102 and 104, respectively. In an exemplary embodiment, the measured physiologic value may be a user's heart rate. The step of correlating measured heart rate information and measured exercise parameters is carried out by performing a predetermined training routine or exercise session. The exercise session or training routine is carried out by providing a user with a substantially standard heart rate strap as a physiologic monitor 102, like that known in the art for detecting an instantaneous heart rate reading. The heart rate strap is worn across the user's chest and includes means for transmitting the sensed heart rate. The heart rate strap may include a wired connection with the computer or other device, or alternatively, it may include means for wirelessly transmitting its heart rate information to the computer or other device.

Contemporaneously with the sensing of the physiologic value, one or more monitors 104 are configured to generate exercise parameter values over time. For example, when the monitor 104 is a power monitor, the user's power output may be measured using a device such as a PowerTap® power meter on a bicycle wheel. System 100 may further be configured to include an oxygen consumption monitor as a second monitor 104, where the user's oxygen consumption rate may be measured using a device adapted for measuring oxygen consumption. Carbon dioxide exhalation rate may be measured using a similar device. Although exemplary exercise parameter monitoring devices are shown, it should be understood that monitor 104 may be used to measure any measurable exercise parameter.

The exercise session or training routine of step 206 is designed to require that the user perform a sequence of activities involving physical exertion that varies the user's heart rate, while the user is contemporaneously having his or her actual exercise parameters measured. For example, the predetermined training activity or exercise session may include riding a training bicycle having a power meter interconnected therewith for measuring the user's power output. Alternatively, power may be monitored using an exercise device such as an elliptical machine, a treadmill, a rowing machine, etc.

In one embodiment of step 206, the user runs on a treadmill while measuring their speed and heart rate. In order to ensure the accuracy of the received measurement, system 100 may further be configured to contemporaneously record the slope setting on the treadmill. In this way, the user's heart rate reading may be correlated with specific training regimens depending on a given speed and slope. This information may be useful in assessing an individual's weaknesses in a given activity. That is, it may be useful in determining under what conditions the user requires further training such as a given slope and speed combination.

In another embodiment of step 206, the user may run on a treadmill with the slope of the treadmill set at a fixed incline, such as 0 degrees, while measuring his or her heart rate. In this way, the user may correlate a particular speed with a particular heart rate reading such that, during subsequent runs, he or she may estimate his or her speed for a flat piece of track or land based on his or her measured heart rate.

In another embodiment of step 206, the user may use a GPS device configured to measure and/or record speed data, elevation gain, and heart rate. This information will enable the user to calibrate a given speed and/or elevation with a given heart rate reading such that in subsequent training activities the user may estimate his or her speed or elevation gain given a detected heart rate reading.

In one exemplary training exercise for step 206, the predetermined training routine is performed over a period of at least fifteen minutes while the user's heart rate and exercise parameter data is simultaneously monitored and collected. It is understood that the period of time over which the training routine or exercise session is performed may be any desired length and may vary substantially. The predetermined training routine or exercise session may include a sequence of gradual exertion increases throughout the course of the training routine or exercise session. Of course, the training routine or exercise session may include any number of different combinations of sequences. For example, the training routine or exercise session may include a sequence of sharp increases in exertion followed by periods of moderate or little exertion, i.e., coasting.

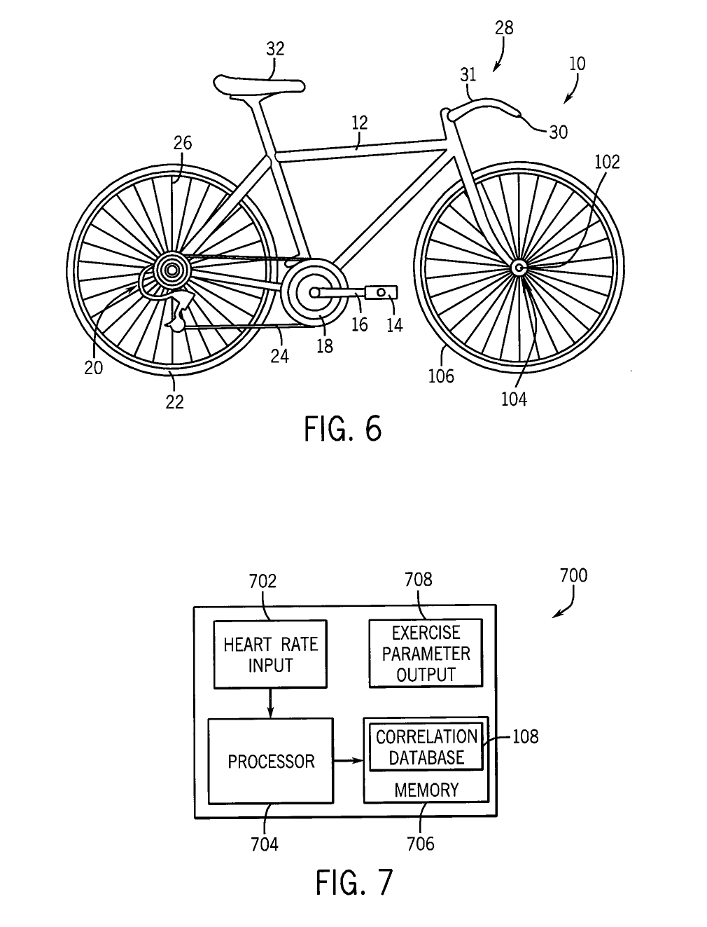

Data generated by monitors 102 and 104 may be transmitted to processor 110 for processing by analysis engine 106 in steps 208-212, depending on the number and/or type of exercise parameter monitors 104 being used with system 100. The data may be transmitted contemporaneously with measurement, and/or may be stored within local memory associated with the respective monitor. Alternatively, the data may be transmitted to a computer or other device having memory to store the heart rate information. The computer or other device may be mounted to a piece of exercise equipment such as the handlebars of a training bicycle as described below with reference to FIG. 6, may be worn by the user on an arm band or the like, or may simply be in the computer in the same general area as the user. Where the information is stored, the data may be transmitted to processor 110 in a subsequent operation.

Following generation of the data and/or contemporaneously during the generation of the data, processor 110 may be configured to receive subjective data from the user of system 100 in a step 214. The subjective information may include a perceived level of effort, which in one embodiment may be used as a substitute for heart rate since evidence exists that it is accurate for determining exercise response than heart rate in some individuals. Perceived exertion could also be used in addition to heart rate to enhance the algorithm and add robustness to the heart rate-based calculation.

Following generation of the data and/or contemporaneously during the generation of the data, processor 110 may be configured to implement a regression analysis engine 106 to generate one or more correlations between the received physiologic data and the one or more exercise parameter values in a step 216. The regression analysis may include any of a variety of known techniques for modeling and analyzing several variables for the purposed of identifying the relationship between a dependent variable, i.e. the measured physiologic value, and one or more independent variables, i.e. the one or more exercise parameter values. The regression analysis may further include modifying the identified relationship based on one or more other input values, such as the user data received in step 202, the skewing factors received in step 204, the subjective data received in step 206, etc.

In any event, the predetermined training routine or exercise session is configured and adapted for determining a correlation between a particular user's measured heart rate and measured parameter such that in subsequent training sessions, the user's measured heart rate may be used to estimate a the parameter such that use of an actual measuring device or system is not necessary. Upon completion of the predetermined training routine or exercise session, the measured heart rate and measured exercise parameter data is compared and plotted with respect to time. In particular, the data measured at the heart rate strap and the exercise parameter measuring device is uploaded to a computer.

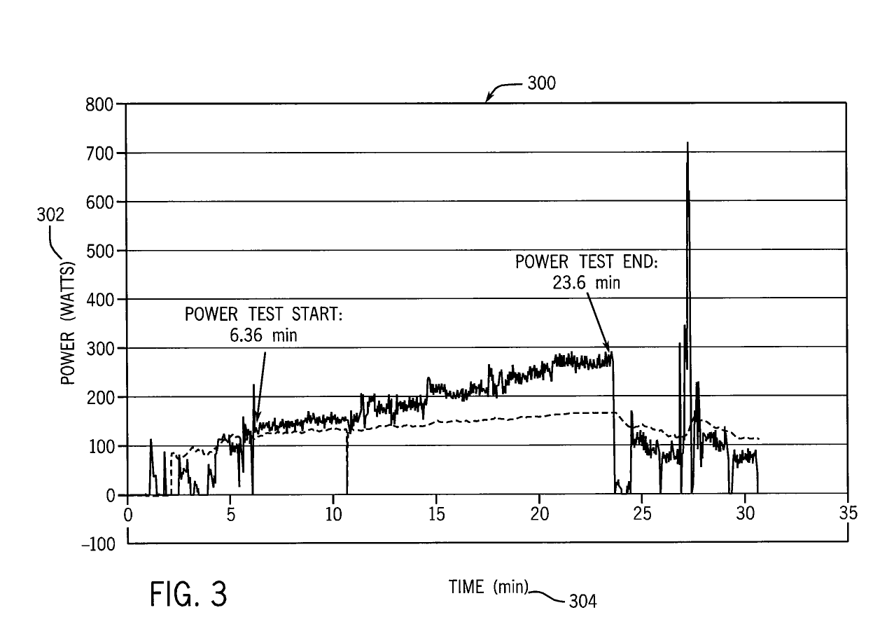

Referring now to FIG. 3, a graph 300 illustrating data collected using an exemplary system 100 configured to receive heart rate data from a monitor 102 and power output data from a monitor 104 is shown. The graph 300 illustrates collected power data, shown with reference to power axis 304, plotted vs. time, shown with reference to time axis 304. This data may be received by processor 110 during the generation of the data or following completion of the calibration activity described with reference to FIG. 2.

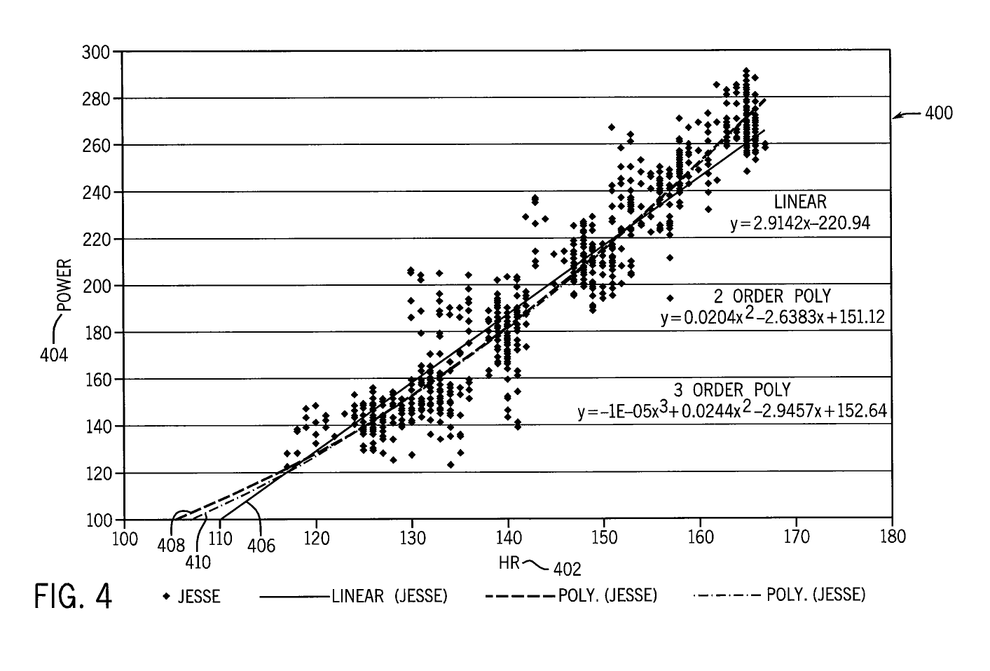

Referring now to FIG. 4, a graph 400 depicting the measured physiologic value 402 against the measured exercise parameter value 404 illustrates the data used by regression engine 106 to generate correlation information. In the example shown in FIG. 4, the measured heart rate information is plotted against measured power on a graph. Specifically, the two measured values are correlated using a (a) linear curve 406, (b) 2nd order polynomial 408, and (c) 3rd order polynomial 410. A representative illustration of the resultant linear, 2nd order, and 3rd order correlation curves with associated calculated correlation constants are shown as curves 406, 410, respectively.

In particular, the linear plot, second order plot, and third order plot are calculated according to the following equations where a, b, c, and d are unique calibration parameters and x is the user's heart rate. The user's heart rate may be an instantaneous heart rate measurement at any given point in time, an average heart rate, or may be an average, a median or mode heart rate over a defined period of time.

Linear: Power=ax+b

Second Order: Power=ax2+bx+c

Third Order: Power=ax3+bx2+cx+d

As noted previously, FIG. 4 represents an exemplary plot showing the linear, second order, and third order power output correlation plots. Accordingly, using the data as shown in FIG. 4, regression analysis engine 106 can generate the variables a, b, c, and d to thereby establish an appropriate correlation between, in this example, the user's heart rate and power output.

In an alternative embodiment of the invention, a differential correlation may be performed by the regression analysis engine 106 to increase the accuracy of the estimated power output calculation. Differentiating the heart rate data with respect to time allows for a more accurate correlation and significantly less lag between the measured heart rate and the estimated power output. The correlation generated by regression analysis engine 106 may be further improved by determining whether the user's heart rate is increasing or decreasing at a particular point in time. For purposes of the differential calibration, additional training routines or exercise sessions may be performed for establishing the differential correlation. For example, a series of sprints may be performed such that the user has to suddenly increase his or her power output. Each of the sprints will then be followed by a time at rest, e.g. between 2-5 minutes. Thus, the data captured from the series of sprints and rest periods may be used to correlate the rate of increase or decrease in measured heart rate to the user's actual power output. A general equation for the differential calibration is as follows, where x is heart rate and a, b, c, and d are unique user calibration parameters.

Linear with Differential: Power=ax+b+c(dx/dt) (where (dx/dt) may be instantaneous or based on a running average)

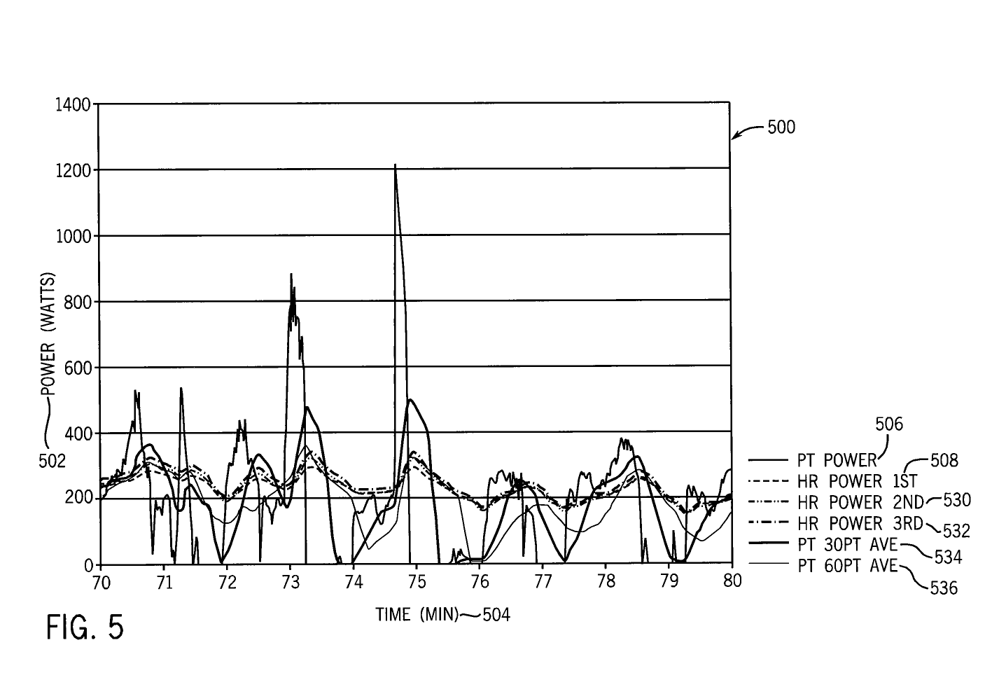

Referring now to FIG. 5, a graph 500 illustrating the differential calculation plots for power output 502 over time 504 is shown. Similar to the calculation described with respect to FIG. 3, regression analysis engine 106 may utilize the linear plot of power output 506 with differential plots 508-512 of first, second, and third order differentials, respectively, to determine the user-specific constants a, b, and c. Further, as seen in FIG. 5, the linear differential correlation enables the development of a plot, indicating the relationship identified by regression analysis engine 106 that mirrors a standard power output plot as a function of time. In this way, not only can a user determine or estimate his or her instantaneous power, but he or she can see how his or her power output fluctuates over the course of a workout and determine average power during all or any selected portion of a workout.

Referring again to FIG. 1 and FIG. 2, in a step 218, system 100 may be configured to store the identified correlation relationship between the measured physiologic data and the measured exercise parameter data in a correlation database 108 in memory 112. The identified relationship may further be indexed based on measured, detected, and/or manually entered data, such that the identified relationship may be utilized based on subsequent detection of similar data as further described below.

In one embodiment, a default correlation may be substituted for the performance of the predetermined training routine or exercise session. This is particularly advantageous for users who may not have access to an actual power meter or similar exercise parameter measuring device necessary for performing the correlation. The default correlation uses known physiological parameters for a particular user and applies them to develop a correlation consistent with other users having similar physiological parameters. Thus, the user of the apparatus and method according to the invention is able to access the basic functionality of the apparatus and method immediately without having to go through the predetermined training routine or exercise session.

Another type of correlation may be calculated based on a user's fatigue level. In this correlation, a standard correlation may be performed using the predetermined training routine or exercise session or alternatively using the default correlation prior the beginning of a lengthy workout. At the end of the workout, the user performs the standard correlation provided by the predetermined training routine or exercise session. In this embodiment, the amount of energy expended is calculated during the actual workout. The energy expended is then compared to the actual heart rate and power data. In this way, by comparing the correlation curve with the total energy expended during the workout, the method and apparatus of the invention can better account for fatigue and improve the correlation between heart rate and power output throughout the course of the workout itself.

Yet another type of correlation may be performed in which the user performs the predetermined training routine or exercise session while periodically entering an assessment of his or her perceived effort expenditure at predetermined points during the training routine or exercise session. The subjective effort expenditure assessment data may be entered at the display device or other such means. After the end of the training routine or exercise session, the data is compared with the user's subjective effort expenditure data to allow for increased accuracy between a user's perceived effort expenditure, heart rate, and actual exercise parameter, e.g. power output.

Referring now to FIG. 6, a representative application of the present invention involves a bicycle 10 that includes a frame 12 that rotatably supports a pair of pedals 14 connected by crank arms 16 to a chain ring 18. The chain ring 18 is coupled to the hub 20 of the rear wheel 22 by a chain 24. The bicycle 10 is powered by a cyclist providing rotational forces to the chain ring 18 via the pedals 12 and crank arms 14. The rotation of the chain ring 18 is transferred by the chain 24 to the rear wheel hub 20, which carries the rear wheel 22 into rotation via spokes 26 to drive the bicycle 10 into motion. Advantageously, and in accordance with the present invention, the bicycle 10 need not include hardware for monitoring exercise parameters.

A computer 28 is mounted to a pair of handlebars 30 at a front end of bicycle 10. Computer 28 includes a display screen 31 for communicating to the user his or her relevant performance data such as heart rate, distance traveled, elevation and speed (all of which are conventional), and, in accordance with the present invention, estimated values of one or more of oxygen consumption, carbon dioxide exhalation, and/or power output. Computer 28 includes means for receiving data from a heart rate sensor, such as a chest strap 32. Computer 28 further includes a processor configured for storing and processing the data received from the transmitter according to the method of the present invention as discussed previously.

Referring now to FIG. 7, an exemplary computer 700 configured to generate one or more estimated exercise parameters based on a detected physiologic value, heart rate in this exemplary embodiment, is shown. Computer 28 may be configured to include a heart rate input device 702, a computer processor 704, memory 706 configured to include a correlation database 108, generated as described above, and an exercise parameter output data 708. Computer 28 may be configured to include more, fewer, and or a different arrangement of components to implement the functions described herein. Computer 700 may be any device configured to utilize a measured physiologic value, such as a heart rate, to determine an estimate exercise parameter value based on stored correlation values.

Input device 702 may be a heart rate sensor, a receiver configured to receive a sensed heart rate value, etc. configured to provide a sensed heart rate value to processor 704. Similarly, output device may be a display, a transmitter configured to transmit and estimate exercise parameter value, etc. configured to provide the estimated value generated by processor 704. Correlation database 108 may be populated with a number of different correlation values to be used based on a variety of inputs in addition to heart rate input device 702. For example, database 108 may be configured to include a default correlation that will provide an estimated power output value for an average user based on heart rate input data 702, an estimated power output value for an average user tailored based on one or more input values such as height, weight, gender, fitness level, workout history, heart rate, fatigue level, etc., a correlation specific to the user, etc.

Computer 700 may be implemented in several different embodiments to perform the functions described herein. In a first embodiment of the invention, computer 28 may be implemented as a component in a heart rate sensor. The heart rate sensor strap is worn by the user and collects heart rate data as is generally understood. In this embodiment, the collected heart rate data 702 is generated locally and is used by processor 704 in combination with stored correlation data in database 108, to provide an output that includes an estimate of one or more exercise parameters, e.g. power output, oxygen consumption, carbon dioxide exhalation, etc. The exercise parameter may be provided using exercise parameter output data 708, which in this embodiment may be a signal representing both sensed heart rate and calculated exercise parameter data. The signal may be transmitted to, for example a bicycle computer 28 as described above. In this manner, the bicycle computer can display sensed heart rate values, estimated exercise parameter values, or both.

In this embodiment, the heart rate sensor strap is configured with electronics for supporting the correlation data calculation and application. Once the user-specific correlation data is stored, the processor is configured for calculation of the estimated exercise parameters during subsequent training sessions as is readily appreciated. Further, the heart rate sensor strap includes a transmitter for transmitting the calculated heart rate data as well as the estimated exercise parameter data to a receiver. The receiver may be a computer such as a cycling computer, a dedicated activity computer adapted to display the transmitted data on a display screen and/or any number of hand-held or user worn computers capable of receiving and displaying such data. In this manner, the user is not limited to obtaining exercise parameter data, such as power data when cycling, but may also obtain such data while conducting other exercise activities as noted previously, many of which heretofore involve activities in which any type of objective exercise parameters simply cannot be measured. The modified heart rate sensor according to this embodiment further may include means for storing the measured heart rate data and calculated estimated exercise parameter data for use after the completion of the specified exercise activity. In this manner, not only can the user monitor his or her instantaneous or near-instantaneous exercise parameters, but the user can use the collected data after the fact to assess his or her performance as compared to, for example, previous exercise activities.

In a second embodiment, computer 700 may be integrated into an in-session computer 28 of the kind generally known in the art, such as a bicycle computer, a wrist-mounted running computer, a computer associated with a piece of stationary exercise equipment, etc. As is generally recognized, such computers are commonly mounted to the item of equipment or carried by the user, and include a display for providing various data to the user. For example, cycling computers typically provide users with heart rate, distance, elevation, power output, and other such measured data. In the present embodiment, the calculated correlation data is loaded directly into the computer. The computer is in communication, wired or wirelessly, with the heart rate sensor worn by the user and receives the data collected by the heart rate strap using heart rate input data 702, which may be a receiver in this embodiment. The collected data is processed by processor 704 with respect to the user-specific correlation data in memory 706 for calculating one or more estimated exercise parameters. The estimated exercise parameters calculated by the computer are provided as exercise parameter output data 708 to the user on a display associated with the computer 28 as is generally understood in the art. In this manner, the person need not have the equipment required for detecting such exercise parameters installed on his or her bicycle or other item of equipment, but instead, he or she may simply use the heart rate sensor and a standard dedicated activity computer 28 to obtain an estimated exercise parameter calculation, which can be displayed to the user real-time. In addition, the estimated exercise parameter data may be uploaded from the dedicated activity computer to a personal computer or the like for analysis after the fact.

The computer 700 may include any number of known display devices or other such devices capable of operating to display data as is generally understood. Such known devices include, but are not necessarily limited to, Cycleops Joule 2.0, Cycleops Joule 3.0, athlosoft ERGOCAD, Bontrager Node 1, Bontrager Node 2, Garmin Edge 705, Garmin Forerunner 310XT, Garmin Edge 500, iBike iAero, iTMP SMHEART Link, O-Synce Macro X, O-Synce Macro High X, Schwinn MPower Performance Console, Tacx Bushido T1980, Tacx Bushido Cycle Computer, and Wahoo_Fitness Fisicia for iPhone/iPod Touch, and the like. Any number of other devices may be used for receiving and displaying the exercise parameter data calculated according to the present invention. It can be appreciated that in certain instances, the display devices include means for performing the heart rate to exercise parameter calculation, thus the user may simply use a normal heart rate strap or sensor as in the first and second embodiments.

In a third embodiment, computer 700 may be integrated into a personal computer system configured to receive stored data after the completion of an activity. In this embodiment, heart rate input data 702 data may be received from a stored log in the memory of a heart rate sensor, a bike computer 28, a wrist computer, etc. The correlation data arrived at through one or more the foregoing correlations is stored either locally, i.e., in a program stored on the personal computer, or at an internet-based server. In either case, the collected heart rate data is then uploaded to the computer and analyzed either via the stored program on the personal computer or through an internet-based program in which the user uploads his or her data to a web site. Accordingly, once the data is uploaded, the estimated desired exercise parameters are calculated by processor 704 using the stored heart rate data in memory 706 and according to the various correlations for the user that are stored in the program. The exercise parameter output data 708 may be displayed to the user in a variety of forms, such as a graph depicting estimated power data for a recently completed workout, graphs of several workouts to show progress over time, etc.

The invention employs a heart rate sensor like those known in the art in which the heart rate sensor is worn on or across the user's chest to thereby detect the heart rate of the user. Typically, heart rate sensors of this kind are in communication with a display means for displaying an instantaneous heart rate measurement. The present invention may use a heart rate sensor that operates in much the same manner to communicate data to another device such as a computer or display, but it may further include means for data collection and processing and for broadcasting not only heart rate data as in known heart rate sensor straps but also estimates of one or more other exercise parameters.

Once the correlation of a particular user's heart rate and exercise parameter is employed, the user may utilize the device and method of the present invention to monitor his or her estimated exercise parameters during subsequent workouts. In this manner, in subsequent training sessions, these values are applied to the measured heart rate to obtain an estimated power output. Thus, the user no longer needs to use an actual power meter to obtain power measurements during his or her exercise activities. Further, because a power meter is not necessary, during subsequent activities, the user need not limit himself or herself to bicycling or other equipment on which power is tested, but he or she may determine or estimate power during any number of exercise activities where previously it was impossible or impractical to obtain a measure of power due to the equipment generally required for obtaining these measurements. Advantageously, as can be readily understood, the method of the present invention enables a user to monitor his or her exercise parameters without the use of relatively expensive equipment and in a greater variety of training activities than is allowed by current equipment. For instance, the method of the present invention allows a user to monitor his or her exercise parameters during bicycling (as discussed previously), walking, running, rowing, rock climbing, swimming, weight lifting, and other exercise activities, many of which employ devices that cannot typically be used to measure exercise parameters, or that do not use any devices at all. In addition, for traditional uses such as in cycling, the user may choose to purchase only a single power meter for one of his or her bicycles while relying on estimated power output during rides on his or her other bicycles. Thus, the user does not have to install a power meter on each of his or her bicycles, but he or she may still be able to assess his or her power output irrespective of the particular bicycle they ride during a workout. Furthermore, the present invention is advantageous when a user engages in more than one physical activity at a time, e.g. during duathlon, triathlon or other multi-sport events, so as to estimate total power output, oxygen consumption, carbon dioxide exhalation, etc. throughout all activities rather than only during certain activities.

In addition to the foregoing, the computer 700 may be configured to receive input data from any number of additional components for improving the accuracy of the exercise parameter calculations. For instance, a cadence sensor may be employed for measuring the user's cadence during bicycling to allow for improved correlation between the measured heart rate and the estimated power output. That is, especially in cycling, when the user's cadence is zero, i.e., when he or she is coasting, the rider's power is understandably zero, however, his or her heart rate will not be zero, thus providing a false power output determination. By using a cadence sensor, the present invention can compare the collected cadence information with the collected heart rate information to better determine the estimated power output. Said another way, the method of the present invention enables the display device associated with the method to display zero for power output at times when the user's cadence is zero instead of showing a relatively low power output during that time period thus providing improved instantaneous and average power output readings.

As discussed previously, a GPS receiver may likewise provide input to computer 700. The GPS receiver could be implemented into the computer 28. Known GPS receivers are capable of detecting speed, distance traveled, elevation, and rate of climb and descent. These pieces of data may be used to further increase the accuracy of the estimated exercise parameter data in light of the calculated heart rate as is readily understood.

An inclinometer and/or speed sensor may likewise provide input to computer 700 in which the inclinometer measures the angle of climb or descent. The angle data can then be combined with the speed data to improve the accuracy of the estimated exercise parameter calculation.

In the case of a GPS receiver, inclinometer, speed sensor or the like, any such component may be incorporated into the heart rate sensor that is worn by the user, or into a dedicated activity computer that is carried by the user or an item of equipment operated by the user, etc.

In another application, the algorithm may be improved or enhanced based on aggregate user data. For example, if a central data collection point were created for users of power and heart rate monitors and enough user data was also collected, the data could, in theory, be used to help users of the system set up their calibration based on the heart rate to power relationships of users with similar profiles to themselves.

In another embodiment of the invention, one or more exercise parameters may be used to provide an estimate of another one or more exercise parameters.

While the present application discusses a number of exercise parameters in detail, it is understood that a number of other, similarly correlatable exercise parameters may be used in the present invention. That is, any parameter that may be correlated with a user's detected heart rate, or any other physiologic metric, may be used in practicing the present invention. For instance, during the initial setup of the system of the invention, the user may measure their actual heart rate, power output, oxygen consumption, and carbon dioxide exhalation. After completion of the data collection, one or more of the foregoing measured values may be subsequently used to estimate one or more of the other of the foregoing measured values. For instance, the measured oxygen consumption and heart rate values may be used to subsequently estimate a power output. Other parameters that may be correlated with heart rate in this manner include, but are not limited to, energy expended, which is typically represented as KiloJoules, and fuel (food) consumed, which is typically represented as Calories. Conversely, during subsequent exercise sessions, a user may measure his or her power output to thereby estimate oxygen consumption or carbon dioxide exhalation. A number of alternative combinations may understandably be used in practicing the method and system of the present invention.

Various modes of carrying out the invention are contemplated as being within the scope of the following claims, particularly pointing out and distinctly claiming the subject matter which is regarded as the invention.

We claim:

1. A computer-implemented method for estimating an exercise parameter for a user, comprising the steps of:
receiving a measured physiologic value;
determining an estimated exercise parameter value based on the physiologic value using a stored correlation between a measured physiologic value and a measured exercise parameter value; and
providing the estimated exercise parameter value.

2. The method of claim 1, wherein the physiologic value is a heart rate value and wherein the exercise parameter value is at least one of power output, oxygen consumption, and carbon dioxide exhalation.

3. The method of claim 1, wherein providing the exercise parameter value includes transmitting a signal including the measured physiologic value and the estimated exercise parameter value.

4. The method of claim 1, wherein receiving a measured physiologic value includes receiving a wireless signal including the measured physiologic value.

5. The method of claim 1, further including receiving a user specific data entry wherein determining an estimated exercise parameter value based on the physiologic value includes customizing the estimated exercise parameter based on the user specific data.

6. The method of claim 5, wherein the user specific data is at least one of a gender, age, height, weight, fitness level, and fatigue level.

7. The method of claim 1, wherein the stored correlation between a measured physiologic value and a measured exercise parameter value is generated based on a regression analysis of received measured physiologic values and measured exercise parameter values.

8. A computer system for providing an estimated exercise parameter value, comprising:
a heart rate sensor input device;
a processor configured to receive heart rate information from the heart rate input device and execute a program to determine a correlation between the received heart rate information and estimated values for one or more exercise parameters; and
an estimated exercise parameter output device.

9. The system of claim 8, wherein the estimated exercise parameter output device is further configured to transmit the received heart rate information.

10. The system of claim 8, further including memory for receiving and storing a measured correlation between the received heart rate information and exercise parameter values for a specific user.

11. A computer implemented method for generating a correlation between a measured heart rate and measured power output for a user during exercise:
receiving a measured heart rate value;
receiving a measured power output value;
determining a user-specific correlation between heart rate and power output using the measured heart rate value and the measured power output value; and
storing the user-specific correlation in computer memory.

12. The method of claim 11, wherein the step of determining a user-specific correlation between heart rate and power output is carried out using a regression analysis that includes determining a linear correlation between heart rate and power output.

13. The method of claim 11, wherein the step of determining a user-specific correlation between heart rate and power output is carried out using a regression analysis and includes determining a first order correlation between the heart rate and power output.

14. The method of claim 11, wherein the step of determining a user-specific correlation between heart rate and power output is carried out using a regression analysis and includes determining a linear correlation, a first order correlation, and a second order correlation between the heart rate and power output.

15. The method of claim 14, further comprising applying a differential correlation between the measured heart rate and power output.

16. The method of claim 11, wherein storing the user-specific correlation in computer memory including associating the user-specific correlation with one or more user input values.

17. The method of claim 16, wherein user input values include at least one of is at least one of a gender, age, height, weight, fitness level, and fatigue level.

18. A computer-implemented method for estimating power output by a user, comprising the steps of:
receiving a measured heart rate value;
determining an estimated power output based on the heart rate value using a stored correlation between a measured heart rate value and a power output value; and
providing the power output value.

19. The method of claim 18, further including receiving a user specific data entry wherein determining an estimated power output value based on the heart rate value includes customizing the estimated power output value based on the user specific data, wherein the user specific data is at least one of a gender, age, height, weight, fitness level, and fatigue level.

20. The method of claim 18, wherein the stored correlation between a measured heart rate value and a power output value is generated based on a regression analysis of received measured heart rate values and power output exercise parameter values.
